(19) United States
(12) Patent Application Publication
(10) Pub. No.: US 2025/0378951 A1
(43) Pub. Date: Dec. 11, 2025

(54) SYSTEMS AND METHODS FOR ASSESSING BLOOD PERFUSION

(71) Applicant: BARD PERIPHERAL VASCULAR, INC., Franklin Lakes, NJ (US)

(72) Inventors: Thomas Ferari, Hudson Falls, NJ (US); Andrew Moll, Portland, OR (US); Alexander Palmer, Gilbert, AZ (US); Olivia R. Palmer, Nashville, TN (US); Oladipo Peter Akerele-Ale, Phoenix, AZ (US); Breanna Simpson, Phoenix, AZ (US)

(73) Assignee: BARD PERIPHERAL VASCULAR, INC., Franklin Lakes, NJ (US)

(21) Appl. No.: 18/876,211

(22) PCT Filed: Jul. 21, 2022
(86) PCT No.: PCT/US2022/037862

§ 371 Date: Dec. 18, 2024

(51) Int. Cl. G16H 50/20; A41D 1/00; A61B 5/00; A61B 5/0205; G16H 10/60; G16H 40/63

(52) U.S. Cl. CPC G16H 50/20; A41D 1/002; A61B 5/02055; A61B 5/6804; G16H 10/60; G16H 40/63

(57) ABSTRACT

Systems and methods for assessing blood perfusion include a wearable garment with a plurality of sensors affixed, a processor communicatively coupled to the sensors, a memory component communicatively coupled to the processor, and machine-readable instructions causing the processor to perform operations including receiving a first set of blood perfusion metrics associated with an individual wearing the wearable garment from the plurality of sensors, generating a first reading based on the first set of blood perfusion metrics, receiving a second set of blood perfusion metrics associated with the individual wearing the wearable garment from the plurality of sensors, generating a second reading based on the second set of blood perfusion metrics, determining an intervention perfusion status based on the first reading and the second reading, and generating, with the machine learning model, an intervention recommendation based on the first reading, the second reading, the intervention perfusion status, or combinations thereof.

### TECHNICAL FIELD

The present disclosure relates to systems and methods for assessing blood perfusion, and more particularly to systems and methods including wearable garments for assessing blood perfusion of a body part wearing the wearable garment.

### BACKGROUND

Blood perfusion is the flow of blood through the vasculature and is responsible for the transport of oxygen, nutrients, waste, and the like throughout the body. Though generally related to blood flow, proper blood perfusion through the periphery of the body is important for the proper functioning of the body. For example, peripheral artery disease (PAD) may reduce blood perfusion to the legs and, occasionally, the arms. Due to reduced blood perfusion to the extremities, PAD may cause complications in the extremities and, in extreme cases, may lead to gangrene and amputation.

Treating PAD is often not straightforward. An ankle-brachial index (ABI) may help diagnose PAD, but it does not provide spatial resolution on potential perfusion problem areas. In addition, vessel spasms related to PAD may impede an initial angiogram. When PAD is treated, the success of PAD interventions is a subjective determination, which may lead to improper or omitted follow-up intervention. Moreover, current devices for determining blood perfusion are expensive, complicated, and provide limited follow-up for monitoring perfusion success after medical intervention. Therefore, devices, systems, and methods are desired for quantifying blood perfusion in extremities for patients undergoing limb revascularization to provide an improved metric for intervention success.

### SUMMARY

In accordance with one embodiment of the present disclosure, a system for assessing blood perfusion includes a wearable garment, a plurality of sensors affixed to the wearable garment, a processor communicatively coupled to the plurality of sensors, a memory component communicatively coupled to the processor, a machine learning model stored in the memory component, and machine-readable instructions stored in the memory component. The machine-readable instructions cause the processor to perform operations including receiving a first set of blood perfusion metrics associated with an individual wearing the wearable garment from the plurality of sensors, generating a first reading based on the first set of blood perfusion metrics, receiving a second set of blood perfusion metrics associated with the individual wearing the wearable garment from the plurality of sensors, generating a second reading based on the second set of blood perfusion metrics, determining an intervention perfusion status of a medical intervention to improve blood perfusion for the individual based on the first reading and the second reading indicative of a level of blood perfusion improvement, and generating, with the machine learning model, an intervention recommendation indicative of whether additional intervention is recommended based on the first reading, the second reading, the intervention perfusion status, or combinations thereof.

In accordance with another embodiment of the present disclosure, a system for assessing blood perfusion, includes a processor, a memory component communicatively coupled to the processor, a machine learning model stored in the memory component, and machine-readable instructions stored in the memory component. The machine-readable instructions cause the processor to perform operations including receiving a first set of blood perfusion metrics associated with an individual from a wearable device having a plurality of sensors for assessing blood perfusion when the individual is wearing the wearable device, generating a first reading based on the first set of blood perfusion metrics, receiving a second set of blood perfusion metrics associated with the individual from the wearable device when the individual is wearing the wearable device, generating a second reading based on the second set of blood perfusion metrics, determining an intervention perfusion status for improving blood perfusion for the individual based on the first reading and the second reading and indicative of a level of blood perfusion improvement, and generating, with the machine learning model, an intervention recommendation indicative of whether additional intervention is recommended based on the first reading, the second reading, the intervention perfusion status, or combinations thereof.

In accordance with yet another embodiments of the present disclosure, a method for assessing blood perfusion includes receiving, with a processor, a first set of blood perfusion metrics associated with an individual wearing a wearable device from the wearable device having a plurality of sensors for assessing blood perfusion, generating, with the processor, a first reading based on the first set of blood perfusion metrics, receiving, with the processor, a second set of blood perfusion metrics associated with the individual wearing the wearable device from the wearable device, generating, with the processor, a second intervention reading based on the first set of blood perfusion metrics, determining an intervention perfusion status for improving blood perfusion for the individual based on the first reading and the second reading and indicative of a level of blood perfusion improvement, and generating, with a machine learning model, an intervention recommendation indicative of whether additional intervention is recommended based on the first reading, the second reading, the intervention perfusion status, or combinations thereof.

Although the concepts of the present disclosure are described herein with primary reference to feet, it is contemplated that the concepts may have applicability to any body part. For example, and not by way of limitation, it is contemplated that the concepts of the present disclosure may enjoy applicability to hands.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 schematically depicts an illustrative system including an illustrative wearable device and system modules, according to one or more embodiments shown and described herein;

FIG. 2 schematically depicts the illustrative wearable device of FIG. 1 having a plurality of sensors, according to one or more embodiments shown and described herein;

FIG. 3 depicts a flow chart illustrating a method for providing an assessment of blood perfusion with the system of FIG. 1, according to one or more embodiments shown and described herein;

FIG. 4 depicts an illustrative method for reinforcing a machine learning model, according to one or more embodiments shown and described herein; and

FIG. 5 depicts an illustrative scenario of a subject wearing a wearable device for providing an assessment of blood perfusion with the system of FIG. 1, according to one or more embodiments shown and described herein.

### DETAILED DESCRIPTION

The embodiments disclosed herein are generally directed to systems and methods for providing an assessment of blood perfusion, which is local fluid flow through a capillary network and extracellular spaces of living tissue at a living tissue site of, for example, an individual, to be characterized as a volumetric flow rate per tissue volume at the site. Blood perfusion aids in providing nutrients and removing cellular waste, and a measured low oxygen saturation level may indicate low blood perfusion. The system includes a wearable device that may be a wearable garment that includes a plurality of sensors that may help determine the spatial resolution of potential perfusion problem areas through the determination of at least oxygen saturation levels without the need for medical procedures. The wearable device further includes or communicates with a computing system for analyzing the data gathered by the plurality of sensors. The wearable device and/or the system may have machine-readable instructions for quantifying the perfusion of the body part wearing the wearable garment based on the gathered data. This, in turn, may make the determination of the success of PAD interventions more objective. The machine-readable instructions may further include a machine learning model that is trained on sensor data for a variety of patients having PAD interventions of varying degrees of success. The machine learning model may further provide follow-up monitoring for determining perfusion success after intervention. Accordingly, systems and methods as described herein provide intuitive means for determining and/or monitoring blood perfusion.

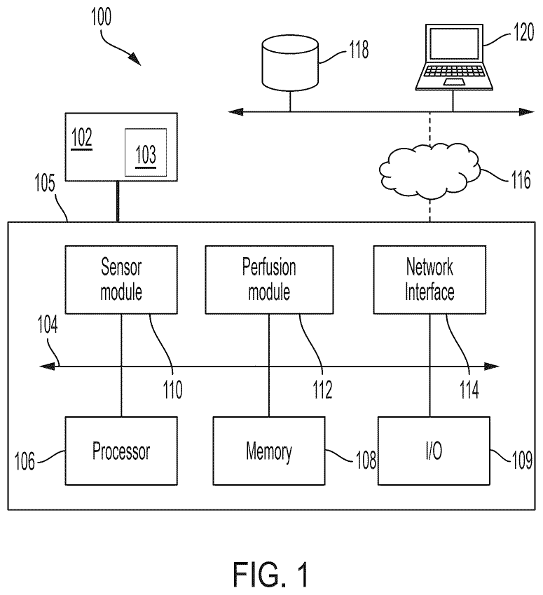

Referring now to FIG. 1, a system 100 including a wearable device 102 including one or more sensors 103 is schematically depicted. The wearable device 102 may include socks, gloves, sleeves, and/or any other wearable garment for assessing blood perfusion. For example, the wearable device 102 may be configured to be worn on a limb of a subject (e.g., arm, hand, leg, or foot). The system 100 may further include a system control module 105 communicatively coupled to the wearable device 102. One or more modules of the system control module 105 may be disposed in or remote from the wearable device 102. The system control module 105 may include at least a processor 106, a memory 108, an input/output interface (I/O interface 109), a sensor module 110, a perfusion module 112, and a network interface 114. The system control module 105 may further include a communication path 104 for communicatively coupling the various components of the system control module 105.

The processor 106 may include one or more processors that may be any device capable of executing machine-readable and executable instructions. Accordingly, each of the one or more processors of the processor 106 may be a controller, an integrated circuit, a microchip, or any other computing device. The processor 106 is coupled to the communication path 104 that provides signal connectivity between the various components of the system control module 105 and the wearable device 102. Accordingly, the communication path 104 may communicatively couple any number of processors of the processor 106 with one another and allow them to operate in a distributed computing environment. Specifically, each processor may operate as a node that may send and/or receive data. As used herein, the phrase “communicatively coupled” means that coupled components are capable of exchanging data signals with one another such as, e.g., electrical signals via a conductive medium, electromagnetic signals via air, optical signals via optical waveguides, and the like.

The communication path 104 may be formed from any medium that is capable of transmitting a signal such as, e.g., conductive wires, conductive traces, optical waveguides, and the like. In some embodiments, the communication path 104 may facilitate the transmission of wireless signals, such as Wi-Fi, Bluetooth, Near-Field Communication (NFC), and the like. Moreover, the communication path 104 may be formed from a combination of mediums capable of transmitting signals. In one embodiment, the communication path 104 comprises a combination of conductive traces, conductive wires, connectors, and buses that cooperate to permit the transmission of electrical data signals to components such as processors, memories, sensors, input devices, output devices, and communication devices. Additionally, it is noted that the term “signal” means a waveform (e.g., electrical, optical, magnetic, mechanical, or electromagnetic), such as DC, AC, sinusoidal-wave, triangular-wave, square-wave, vibration, and the like, capable of traveling through a medium.

The memory 108 is communicatively coupled to the communication path 104 and may contain one or more memory modules comprising RAM, ROM, flash memories, hard drives, or any device capable of storing machine-readable and executable instructions such that the machine-readable and executable instructions can be accessed by the processor 106. The machine-readable and executable instructions may comprise logic or algorithms written in any programming language of any generation (e.g., 1 GL, 2 GL, 3 GL, 4 GL, or 5 GL) such as, e.g., machine language, that may be directly executed by the processor, or assembly language, object-oriented languages, scripting languages, microcode, and the like, that may be compiled or assembled into machine-readable and executable instructions and stored on the memory 108. Alternatively, the machine-readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components.

The I/O interface 109 is coupled to the communication path 104 and may contain hardware for receiving input and/or providing output. Hardware for receiving input may include devices that send information to the processor 106. For example, a keyboard, mouse, scanner, touchscreen, and camera are all I/O devices because they provide input to the processor 106. Hardware for providing output may include devices from which data is sent. For example, an electronic display, indicator light, speaker, and printer are all I/O devices because they output data from the processor 106.

The sensor module 110 is coupled to the communication path 104 and communicatively coupled to the processor 106. The sensor module 110 may be communicatively coupled to the one or more sensors 103 that may include a plurality of sensors for measuring pulse oximetry (to, for example, measure oxygen saturation (SpO₂) levels as a percentage of hemoglobin binding sites in the bloodstream at a measured site occupied by oxygen as a ratio of oxygen-saturated hemoglobin to total hemoglobin), heart rate, temperature, bioimpedance, and/or other vital signs, as well as statistical confidence percentage of a level of blood perfusion. Accordingly, the one or more sensors 103 of the wearable device 102 may include blood pressure monitors, pulse oximeters, optical heart sensors, thermometers, bioimpedance sensors, and/or the like. The one or more sensors 103 may comprise any suitable sensor configured to measure and quantify the blood perfusion of a patient, or any metric correlated with blood perfusion of a patient, at a location to which the sensor 103 is placed.

The perfusion module 112 may be a hardware module coupled to the communication path 104 and communicatively coupled to the processor 106. The perfusion module 112 may also or instead be a set of instructions contained in the memory 108. The perfusion module 112 may be configured to receive blood perfusion metrics, generate blood perfusion readings, determine intervention statuses (e.g., intervention perfusion status), predict intervention statuses (e.g., predict intervention perfusion status), and/or recommend further courses of treatment. The perfusion module 112 may be further configured to train and utilize a machine learning model for generating predations of blood perfusion and/or recommendations for courses of treatment (e.g., interventions such as medical or surgical interventions). The perfusion module 112 may utilize supervised methods to train a machine learning model as an artificial intelligence (AI) model component that may be disposed in the memory 108 based on labeled training sets, wherein the machine learning model is a decision tree, a Bayes classifier, a support vector machine, a convolutional neural network, and/or the like. In some embodiments, unsupervised machine learning algorithms may be used, such as k-means clustering, hierarchical clustering, and/or the like. The perfusion module 112 may also be configured to perform the methods as described herein.

As noted above, the system control module 105 may also include the network interface 114 communicatively coupled to the communication path 104. The network interface 114 can be any device capable of transmitting and/or receiving data via a network or other communication mechanisms. Accordingly, the network interface 114 can include a communication transceiver for sending and/or receiving any wired or wireless communication. For example, the network interface 114 may include an antenna, a modem, an Ethernet port, a Wi-Fi card, a WiMAX card, a cellular modem, near-field communication hardware, satellite communication hardware, and/or any other wired or wireless hardware for communicating with other networks and/or devices. The network interface 114 communicatively connects the system control module 105 to external systems, such as external computing devices 120, via a network 116. The network 116 may be a wide area network, a local area network, a personal area network, a cellular network, a satellite network, and the like.

The system control module 105 of the system 100 may be communicatively connected to one or more external computing devices 120. The external computing devices 120 may be one or more computing devices that may be in remote communication with the wearable device 102 and the system control module 105 via network 116. In embodiments, the system control module 105 may be a computing device including one or more modules as described herein remote from or integrated within the wearable device 102. The external computing devices 120 may include devices that operate beyond the wearable device 102 such as desktop computers, laptop computers, smartphones, and any other type of computing device in communication with the wearable device 102 and/or the system control module 105. The external computing devices 120 may send to and/or receive from the wearable device 102 and/or the system control module 105 information, such as sensor information from the sensor module 110.

The wearable device 102 and the system control module 105 of the system 100 may be communicatively connected to one or more remote services 118. The remote services 118 may include services that operate beyond the wearable device 102 and may be utilized by or may utilize the wearable device 102, such as external databases, storage devices, servers, computing platforms, and any other type of service. For example, an external database may store sensor information gathered by the sensor module 110.

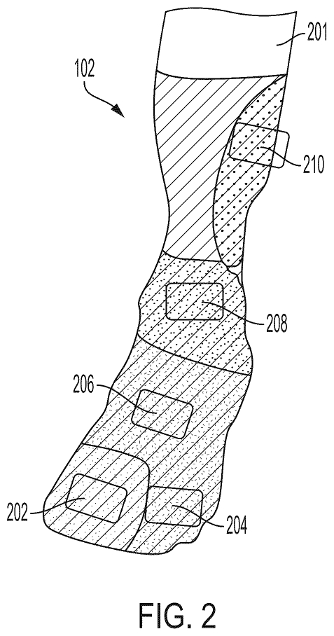

It should be understood that the components illustrated in FIG. 2 are merely illustrative and are not intended to limit the scope of this disclosure. More specifically, while the components in FIG. 2 are illustrated as residing within wearable device 102, this is a non-limiting example. In some embodiments, one or more of the components may reside external to wearable device 102. It should be also be understood that the components of the wearable device 102 and/or the system control module 105 described herein are exemplary and may contain more or less than the number of components shown in FIG. 1. For example, to reduce costs of the wearable device 102, some embodiments of the wearable device 102 may include the sensor module 110 for capturing perfusion metrics to be sent to an external computing device 120 for performing the operations of one or more system control module 105 components and/or methods described herein.

Referring now to FIG. 2, a wearable device 102 of system 100 is depicted. The wearable device 102 may comprise a wearable garment, the one or more sensors 103 of FIG. 1 as a plurality of sensors 202, 204, 206, 208, 210, and may, in some embodiments, include one or more of the other components of the system control module 105 shown in FIG. 1. Costs of the wearable device 102 may be reduced by limiting the wearable device 102 to comprise a wearable garment and a plurality of sensors 202, 204, 206, 208, 210, wherein an external computing device 120 includes the components of the system control module 105 and performs the methods described herein. In these embodiments, the wearable device 102 may be communicatively coupled to an external computing device via a transmitter disposed in the wearable device 102 and a transceiver disposed in the external computing device and configured to receive data from the transmitter). The wearable garment may be a sock, glove, sleeve, and/or any other clothing item. For example, the wearable device 102 is a sock that may be slipped over the foot of a patient 201. The wearable garment of the wearable device 102 may generally be constructed of any type of material, and such materials are not limited by the present disclosure. For example, the wearable garment may be constructed from a textile comprising natural fibers such as, for example, wool, flax, cotton, hemp, and/or the like. In some embodiments, the wearable garment may also or instead be formed from one or more synthetic fibers such as, for example, polyester, aramid, acrylic, nylon, spandex, olefin, carbon fiber, and/or the like. The wearable garment may generally have a variety of sizes to fit properly on patients 201 of different sizes. For example, the wearable garment may be available in small, medium, large, and/or extra-large sizes having various lengths and/or widths according to the size of the patient 201.

Having an appropriately fitting wearable garment for a patient 201 allows the wearable device 102 to have consistent placement of the plurality of sensors 202, 204, 206, 208, 210 between wearable garments of the same size and between wears of the same wearable garment. The sensors 202, 204, 206, 208, 210 are positioned relative to or on the wearable garment at positions corresponding to specific areas on the patient body such that the sensors 202, 204, 206, 208, 210 are positioned adjacent to the patient 201 when the wearable garment is worn by the patient 201. As a result, the wearable garment of the wearable device 102 may have repeatable sensor placement with appropriate pressure caused by the wearable garment to maintain contact and proximity of the sensors 202, 204, 206, 208, 210 to a body part, such as a foot.

In addition, the sensors 202, 204, 206, 208, 210 may be positioned corresponding to regions of a body part such that one or more of the plurality of sensors 202, 204, 206, 208, 210 are dedicated to regional measurements. For example, in embodiments, a foot may be divided into regions such that the forefoot has sensors 202, 204, the midfoot has sensor 206, the hindfoot has sensor 208, and/or the calf has sensor 210. Using a plurality of sensors 202, 204, 206, 208, 210 may aid in minimizing error and improving a signal-to-noise ratio. The sensors 202, 204, 206, 208, 210 communicatively coupled to the sensor module 110 may be clusters of sensors that measure pulse oximetry, heart rate, temperature, bioimpedance, and/or other vitals, as well as statistical confidence percentage of a level of blood perfusion, which taken together can improve the accuracy and reliability of the device.

The plurality of sensors 202, 204, 206, 208, 210 function by providing measurements that may be utilized to establish a baseline of blood perfusion based on the ankle-brachial index (ABI) of the patient 201 and then detecting changes in the blood perfusion in the foot of the patient 201 prior to, during, and/or following intervention. The system control module 105 may process the changes in data from the sensors 202, 204, 206, 208, 210 to determine a status of various regions of the body where the sensors 202, 204, 206, 208, 210 are placed. For example, when oxygen saturation (SpO₂) levels are determined to measure a percentage of hemoglobin binding sites in the bloodstream at a measured site occupied by oxygen (i.e., oxygen-saturated hemoglobin relative to total hemoglobin) by each of the plurality of sensors 202, 204, 206, 208, 210, an image or other visual (e.g., an array of lights) may present an indicator of whether the blood perfusion of the patient 201 is good, poor, or critical. Additionally, when the sensors 202, 204, 206, 208, 210 are placed against multiple areas of the foot, for example, the device can detect regional differences of blood perfusion within the foot. For example, the region covered by sensor 202 may have critical blood perfusion, the regions covered by sensors 204, 206 may have poor blood perfusion, and the regions covered by sensors 208, 210 may have good blood perfusion. By incorporating multiple sensors 202, 204, 206, 208, 210 and analyzing the data produced, the wearable device 102 via the system control module 105 provides both immediate feedback and trend data to assess blood perfusion immediately and over time.

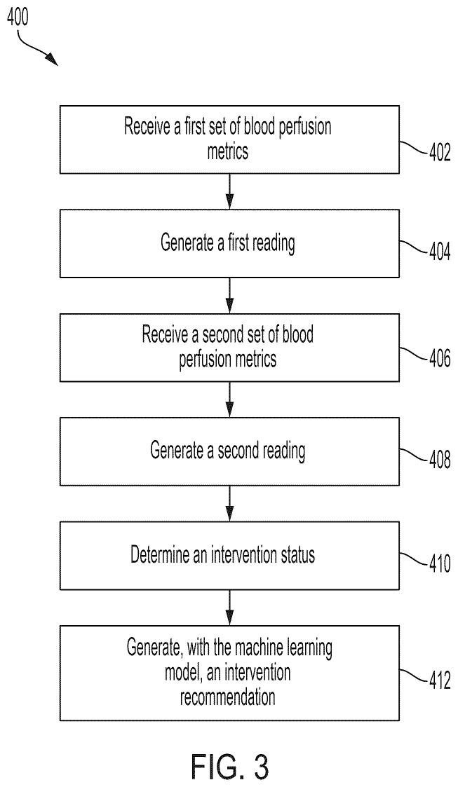

Referring now to FIG. 3, a method 400 as a control scheme that may be implemented by the system 100 and for providing an assessment of blood perfusion with a wearable device 102 is depicted. The method 400 may be performed by the perfusion module 112 of the system control module 105 of FIG. 1. At block 402, the perfusion module 112 of the system control module 105 receives a first set of blood perfusion metrics associated with an individual from the plurality of sensors 202, 204, 206, 208, 210. As discussed above, the system control module 105 includes a sensor module 110 communicatively coupled to the plurality of sensors 202, 204, 206, 208, 210. The plurality of sensors 202, 204, 206, 208, 210 may each be individual sensors or sensor clusters including, but not limited to, blood pressure monitors, pulse oximeters, optical heart sensors, thermometers, bioimpedance sensors, and/or the like. Blood perfusion metrics may be any measurement related to blood perfusion in the body and may be determined at least from information received from the plurality of sensors 202, 204, 206, 208, 210. Accordingly, blood perfusion metrics include blood oxygenation, heart rate, bioimpedance, temperature, ankle-brachial pressure, or combinations thereof. The first set of blood perfusion metrics received may be from one or more regions of the body covered by the wearable device 102. For example, with reference to FIG. 2, the regions may be various parts of the foot. The sensors 202, 204, 206, 208, 210 may gather a first set of blood perfusion metrics associated with an individual (e.g., the patient 201 of FIG. 2) and send the data to the perfusion module 112 of the system control module 105, for example, via the communication path 104.

Referring back to FIG. 3, at block 404, the wearable device 102 generates a first reading based on the first set of blood perfusion metrics. The data received by the perfusion module 112 may be used to determine a pre-intervention reading for establishing a baseline level of blood perfusion. The perfusion module 112 may correlate the sensor measurements with the determined ABI (or other blood perfusion metric) to define levels of blood perfusion and screen for PAD. The ABI may be determined by measuring and selecting a high pressure of two arteries at the ankle and dividing the higher pressure by a brachial atrial systolic pressure at the arm. The measurements used for determining ABI may be received from the wearable device 102 and/or an external measurement device. The reading may include a status of various regions of the body where the sensors 103, 202, 204, 206, 208, 210 are placed. For example, when the SpO₂ levels are determined, a reading may indicate whether patient blood perfusion is good, poor, or critical for each region. As another example, the perfusion module may generate a plot of blood oxygenation (ranging from 0 to 100) to ABI (ranging from 0 to 1.5). Statuses of blood perfusion may be stratified such that, for any blood oxygenation reading, an ABI from 0 to 0.5 is critical, an ABI from 0.5 to 1 is poor, and an ABI from 1 to 1.5 is good. It should be understood that embodiments are not limited to three classifications of blood perfusion and may contain greater or fewer classifications.

Additionally, when the sensors 103, 202, 204, 206, 208, 210 are placed against multiple areas, the wearable device 102 may determine regional differences in blood perfusion between two or more regions of the patient. For example, with reference to FIG. 2, the wearable device 102 may determine a regional difference of blood perfusion between the forefoot having sensor 202, the midfoot having sensor 206, and/or the hindfoot having sensor 208 wherein the regional difference indicates a steady degradation in blood perfusion from the hindfoot to the forefoot.

Referring still to FIG. 3, at block 406, the perfusion module 112 receives a second set of blood perfusion metrics associated with the individual wearing the wearable device 102, that may be a wearable garment, from the plurality of sensors 202, 204, 206, 208, 210. The perfusion module 112 may receive the second set of blood perfusion metrics in a manner similar to block 402. Unlike block 402, block 406 may occur during and/or after an intervention, such as surgery or other medical intervention, to improve blood perfusion in the patient 201.

At block 408, the perfusion module 112 generates a second reading based on the second set of blood perfusion metrics. That is, the data received by the perfusion module 112 of the wearable device 102 may be used to determine the effects of an intervention on a change in blood perfusion such as during the intervention and/or post-intervention reading for establishing a current level of blood perfusion. Generating a second reading may be performed in a manner similar to block 404.

At block 410, the perfusion module 112 determines an intervention status based on the first reading and the second reading. The perfusion module 112 of the wearable device 102 may identify differences between the first reading and the second reading to define an intervention status to improve blood perfusion and indicative of a level of blood perfusion improvement. Differences may be localized differences to identify changes in a particular region over time (e.g., the differences between the first reading and the second reading may be calculated for the same sensor or group of sensors 202, 204, 206, 208, 210). For example, the differences between the first reading and the second reading of the forefoot may be determined. Differences may also or instead be regional differences to identify how a degree of disparity between two regions has changed over time (for example, the change in difference between two or more sensors may be calculated). For example, the differences between the midfoot and the forefoot in the first reading may be compared to the differences between the midfoot and the forefoot in the second reading. The differences between the midfoot and the forefoot in the first reading may indicate that the midfoot has poor blood perfusion and that the forefoot has critical blood perfusion. FIG. 2 may represent a second, post-intervention reading, showing that the area of the forefoot covered by sensor 204 has improved to poor blood perfusion similar to the midfoot. The difference between the first reading and the second reading in this example would be the area covered by sensor 204 in the forefoot, indicating that the intervention has provided only marginal improvements in blood perfusion.

In some embodiments, a machine learning model communicatively coupled to the perfusion module 112 and/or other components of the system control module 105 may be used to determine an intervention status. The machine learning model may be a classifier that engages in unsupervised machine learning algorithms, such as k-means clustering, hierarchical clustering, and/or the like. The machine learning model may analyze the plurality of readings taken along with other reference data to classify the plurality of readings as belonging to various levels of blood perfusion. For example, if the difference in the features of the first and second readings (e.g., blood oxygenation, temperature, bioimpedance, etc.) are similar to the differences in features of readings taken from a patient 201 who has gone through a successful intervention, the second reading may be classified as a success. The data collected by the device may also be used to classify the status of the patient 201. The device may receive data (e.g., ABI, SpO2, bioimpedance, and temperature) from a variety of other patients as well as their classification (e.g., good, poor, or critical blood perfusion) to train the machine learning model to classify patients. Following training, data relating to changes in the blood perfusion in the foot prior to, during, and/or following intervention may be used as inputs to the trained machine learning model to generate an output representing the patient's classification. In some embodiments, the reference data may include prior blood perfusion metrics and/or prior intervention statuses from a plurality of individuals. The plurality of individuals may be from cross-sections of the general population or subsets thereof having similar attributes to the patient 201. For example, if the patient 201 has a particular medical condition, the reference data may be from individuals having the same medical condition.

Referring still to FIG. 3, at block 412, the perfusion module 112 generates an intervention recommendation indicative of whether additional intervention is recommended based on the first reading, the second reading, the intervention status, or combinations thereof. The perfusion module 112 may generate the intervention recommendation with a machine learning model as described herein. The machine learning model may be a decision tree, a Bayes classifier, a support vector machine, a convolutional neural network, and/or the like. Interventions, as discussed herein, may refer to medical procedures including, but not limited to, angioplasty, bypass surgery, atherectomy, thrombolytic procedures, and/or other procedures for treating symptoms and/or conditions stemming from poor blood perfusion.

The data collected by the wearable device 102 and/or one more components of the system control module 105 as described herein may be used as training inputs to the machine learning model to generate predictions regarding the patient's status and need for subsequent intervention. Data may include changes in the blood perfusion of the patient 201 prior to, during, and/or following intervention. In addition to the patient's data, the system control module 105 may receive a historical data set including prior blood perfusion metrics, prior interventions and the type of intervention, prior intervention statuses before, during and after the intervention, or combinations thereof from a plurality of individuals. The historical data set may be used to train the machine learning model for more accurate predictions. The data from other patients may be from cross-sections of the general population. In some cases, the cross-sections of the population may be those with similar attributes to the patient 201. For example, if the patient 201 has diabetes, the machine learning model may be trained with data relating to blood perfusion in the feet of other patients with diabetes.

The intervention recommendation may be an indication of whether, how much additional intervention is needed, and/or when additional intervention will likely be needed. If the second reading is an intra-intervention reading, then the intervention recommendation may be continuing intervention recommendation that includes an indication whether continued intervention should be provided, a predicted intervention status (e.g., a predicted intervention perfusion status), or combinations thereof. In embodiments, a predicted intervention perfusion status may be indicative of a predicted perfusion status result of the medical intervention at or after completion of the intervention. In embodiments, a predicted intervention perfusion status may be indicative of a predicted perfusion status result of the medical intervention within a time period following the intervention. An indication that continued intervention should be provided may be an indication that the current intervention is insufficient for a threshold level of blood perfusion, which may be predetermined or user-defined. For example, while an intervention is being performed, the doctor performing intervention may receive, in real-time, the indication from the wearable device 102 and/or the system control module 105 that more intervening work should be performed before concluding the intervention because some areas are at a critical level. A predicted intervention status may be an indication of the likely result of the current intervention, as demonstrated by the training data on which the machine learning model was trained. For example, while an intervention is being performed, the doctor performing the intervention may receive, in real-time, the prediction from the wearable device 102 and/or the system control module 105 that the current intervention is likely to only improve blood perfusion to part of a foot rather than the entire foot.

If the second reading is a post-intervention reading, then the intervention recommendation is a follow-up intervention recommendation that includes a recommended course of treatment, a predicted intervention status, or combinations thereof. A follow-up intervention recommendation may be a recommended course of treatment subsequent to an intervention. The recommended course of treatment may be based on past cases having similar first readings, second readings, and/or intervention status and courses of treatment that resolved the past cases, as provided to the machine learning model in the training data. The machine learning model could be trained to make course of treatment recommendations based on past case inputs, recommended course of treatments, and results. Past cases within positive results may then be used to recommend similar courses of treatment. Non-limiting example course of treatment recommendations may include additional intervention(s), specific therapies such as massage and/or drug therapies, and similar treatments to address improving circulation and blood perfusion. As a course of treatment recommendation embodiment and non-limiting example, after an intervention is performed, the doctor may receive the indication from the wearable device 102 and/or the system control module 105 that an additional intervention should be performed. A follow-up intervention recommendation may also or instead include a predicted intervention status. The predicted intervention status may be an indication of the likely result of the intervention recently performed, as demonstrated by the training data on which the machine learning model was trained. For example, after an intervention is performed, the doctor may receive the indication from the wearable device 102 and/or the system control module 105 that blood perfusion will likely be at a good status within a week following the intervention.

In some embodiments, the steps of method 400 may be performed by a system 100 that includes the wearable device 102 in which the wearable device 102 may have the plurality of sensors 202, 204, 206, 208, 210 as the one or more sensors 103 (FIG. 1) to collect blood perfusion metrics that are then passed to the system control module 105 and/or an external computing device 120 for processing according to the steps of method 400.

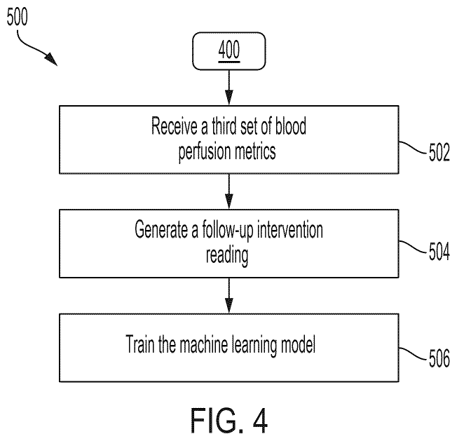

Referring now to FIG. 4, a method 500 for reinforcing a machine learning model is depicted. The method 500 may be performed by the perfusion module 112 of the system control module 105 to enhance the performance of the machine learning model of the perfusion module 112. The method 500 may be a continuation of method 400 to train the machine learning model further. At block 502, the perfusion module 112 receives a third set of blood perfusion metrics associated with the individual wearing the wearable device 102 from the plurality of sensors. The perfusion module 112 may receive the third set of blood perfusion metrics in a manner similar to blocks 402 and 406 of FIG. 3. Unlike blocks 402 and 406, block 502 may occur after a period of recovery from an intervention, such as surgery, to determine the accuracy of a previous prediction of the machine learning model and improve the training of the machine learning model for future predictions.

At block 504, the wearable device generates a third reading as a follow-up intervention reading after generation of the intervention recommendation and based on the third set of blood perfusion metrics. The third reading may be a follow-up reading subsequent to an intervention to determine the accuracy of a predicted intervention status previously made by the machine learning model by comparing the third reading to the predicted intervention status previously made by the machine learning model. If the comparison is within a certain threshold, a certain level of accuracy is determined and quantified. Generating the third reading may be performed in a manner similar to blocks 404 and 408 of FIG. 3.

Referring still to FIG. 4, at block 506, the system control module 105 such as via the perfusion module 112 further trains the machine learning model based on a comparison of the intervention recommendation and the third reading to improve subsequent follow-up intervention recommendations. The intervention recommendation and/or the third reading may be incorporated into the training data used to train the machine learning model. The machine learning model may be re-trained on the updated training dataset. For example, the machine learning model may determine that the comparison of the intervention recommendation and the third reading may be greater than an allowable predetermined threshold. In these embodiments, the machine learning model may be trained, based on the third reading, such that subsequent follow-up intervention recommendations are within the allowable predetermined threshold. In these embodiments, the machine learning model may be trained by adjusting or otherwise changing the parameters and associated weights in the machine learning model to increase model accuracy, so that that subsequent follow-up intervention recommendations are within the allowable predetermined threshold. In embodiments, the updated machine learning model may be validated by comparisons to additional readings taken after subsequent follow-up intervention recommendations that are within an acceptable threshold.

In some embodiments, the steps of method 500 may be performed by a system 100 that includes the wearable device 102 in which the wearable device 102 may have the plurality of sensors 202, 204, 206, 208, 210 as the one or more sensors 103 (FIG. 1) to collect blood perfusion metrics that are then passed to the system control module 105 and/or an external computing device 120 for processing according to the steps of method 500.

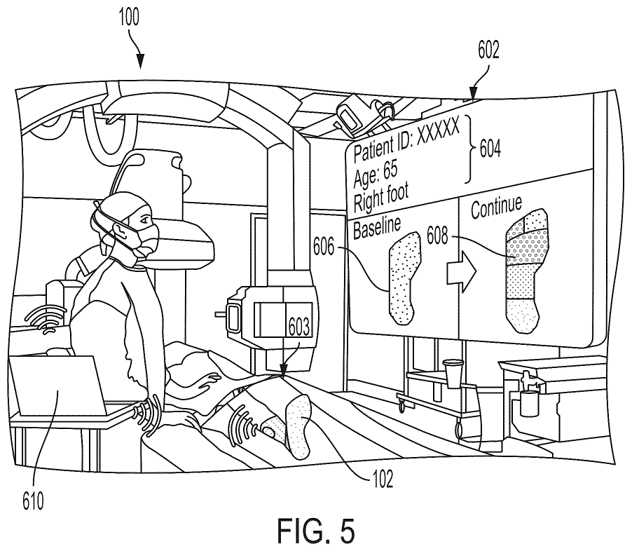

Referring now to FIG. 5, a scenario of a patient 603 wearing a wearable device 102 for providing an assessment of blood perfusion is depicted. A system 100 for assessment of blood perfusion may include the wearable device 102 (that may include one or more components of the system control module 105), an external computing device 610 (that may, additionally or alternatively, include one or more components of the system control module 105), and an electronic display 602. The wearable device 102 may be worn by the patient 603 before, during, and/or after intervention for PAD, for example. The wearable device 102 may be collecting data via the one or more sensors 103 (FIG. 1) such as pulse oximetry, heart rate, temperature, bioimpedance, and/or other vital signs (collectively “blood perfusion metrics”). The raw data collected by the wearable device 102 may be processed into readings by the system control module 105 indicating a level of blood perfusion. For example, ABI, pulse oximetry, and bioimpedance may each be used to screen for PAD. A first set of perfusion metrics may be processed by the perfusion module 112 of the system control module 105 to generate a first reading 606. In an embodiment, the first reading 606 may be transmitted from the system control module 105 to the external computing device 610, which may store, relay, interpret, and/or otherwise utilize the perfusion metrics and/or readings 606, 608 from the system control module 105. For example, the external computing device 610 may relay the readings 606, 608 onto an electronic display 602. In some embodiments, the wearable device 102 only collects perfusion metrics via the one or more sensors 103 and transmits the data to the external computing device 610 including the system control module 105 to perform the rest of the process, such as generating the readings 606, 608. This may reduce cost of the wearable device 102 by placing the computational load onto an external computing device 610. In other embodiments, the wearable device 102 may include one or more components of the system control module 105 such as a processor 106 and/or memory 108 coupled thereto to provide computation power.

During an intervention, the doctor operating on the patient 603 may view, on an electronic display 602, the patient information 604, the first reading 606, and the second reading 608. The first reading 606 may show a level of blood perfusion to establish a baseline, which here shows a critical level of blood perfusion throughout the foot. During the intervention, the system control module 105 may also gather a second set of blood perfusion metrics and determine a second reading 608 of blood perfusion. The second reading 608 of FIG. 5 may indicate that the hindfoot has improved significantly, the midfoot has improved moderately, and the forefoot has improved slightly since the intervention began. However, the second reading 608 may also indicate that the area around the big toe of the foot has not improved. The wearable device 102 may identify these differences between the first reading 606 and the second reading 608 to define an intervention status. Differences may be localized, such as the big toe area of the second reading 608, and may also be regional, such as greater improvement towards the hindfoot.

The system control module 105 may generate the intervention recommendation with a machine learning model communicatively coupled to the perfusion module 112. The machine learning model may be trained on a data set having first readings and second readings of the device for a plurality of interventions performed on prior patients, and one or more blood perfusion metrics describing the patient outcome for each intervention. A threshold level of blood perfusion may exist such that the system control module 105 is configured to prefer a level of blood perfusion above a critical level. Accordingly, the system control module 105 may recommend that the doctor continue intervention, due to the lack of improvement throughout the entirety of the foot. Recommendations may provide doctors with a more objective, data-driven approach to determining the likelihood of success of an intervention for treating PAD. In some embodiments, the generation of the intervention recommendation may be performed by the system 100. For example, the external computing device 610 may generate the intervention recommendation.

Embodiments May be Further Described with Respect to the Following Numbered Clauses:
1. A system for assessing blood perfusion, comprising: a wearable garment; a plurality of sensors affixed to the wearable garment; a processor communicatively coupled to the plurality of sensors; a memory component communicatively coupled to the processor; a machine learning model stored in the memory component; and machine-readable instructions stored in the memory component that cause the processor to perform operations comprising: receiving a first set of blood perfusion metrics associated with an individual wearing the wearable garment from the plurality of sensors; generating a first reading based on the first set of blood perfusion metrics; receiving a second set of blood perfusion metrics associated with the individual wearing the wearable garment from the plurality of sensors; generating a second reading based on the second set of blood perfusion metrics; determining an intervention perfusion status of a medical intervention to improve blood perfusion for the individual based on the first reading and the second reading and indicative of a level of blood perfusion improvement; and generating, with the machine learning model, an intervention recommendation indicative of whether additional intervention is recommended based on the first reading, the second reading, the intervention perfusion status, or combinations thereof.
2. The system of clause 1, wherein the first set of blood perfusion metrics and the second set of blood perfusion metrics include blood oxygenation, heart rate, bioimpedance, temperature, ankle-brachial pressure, or combinations thereof.
3. The system of any preceding clause, wherein the plurality of sensors are positioned on the wearable garment such that the plurality of sensors are positioned adjacent to the individual when the wearable garment is worn by the individual.
4. The system of any preceding clause, wherein: the first reading is a pre-intervention reading for establishing a baseline reading of blood perfusion; the second reading is an intervention reading for establishing a current reading of blood perfusion during the medical intervention; and the intervention recommendation is a continuing intervention recommendation that includes an indication whether continued intervention should be provided, a predicted intervention perfusion status indicative of a predicted perfusion status result of the medical intervention at or after completion of the intervention, or combinations thereof.
5. The system of any preceding clause, wherein: the first reading is a pre-intervention reading for establishing a baseline reading of blood perfusion; the second reading is a post-intervention reading for establishing a current reading of blood perfusion; and the intervention recommendation is a follow-up intervention recommendation that includes a recommended course of treatment, a predicted intervention perfusion status indicative of a predicted perfusion status result of the medical intervention within a time period following the medical intervention, or combinations thereof.
6. The system of any preceding clause, wherein the machine-readable instructions cause the processor to perform operations further comprising: receiving a third set of blood perfusion metrics associated with the individual wearing the wearable garment from the plurality of sensors; generating a third reading as a follow-up intervention reading after generation of the intervention recommendation and based on the third set of blood perfusion metrics; and training the machine learning model based on a comparison of the intervention recommendation and the third reading to improve subsequent follow-up intervention recommendations.
7. The system of any preceding clause, wherein the machine-readable instructions cause the processor to perform operations further comprising, before generating the intervention recommendation, receiving a historical data set including prior blood perfusion metrics, prior interventions, prior intervention statuses, or combinations thereof from a plurality of individuals.
8. The system of clause 7, wherein the machine-readable instructions cause the processor to perform operations further comprising, before generating the intervention recommendation, training the machine learning model based on the historical data set.
9. A system for assessing blood perfusion, comprising: a processor; a memory component communicatively coupled to the processor; a machine learning model stored in the memory component; and machine-readable instructions stored in the memory component that cause the processor to perform operations comprising: receiving a first set of blood perfusion metrics associated with an individual from a wearable device having a plurality of sensors for assessing blood perfusion when the individual is wearing the wearable device; generating a first reading based on the first set of blood perfusion metrics; receiving a second set of blood perfusion metrics associated with the individual from the wearable device when the individual is wearing the wearable device; generating a second reading based on the second set of blood perfusion metrics; determining an intervention perfusion status for improving blood perfusion for the individual based on the first reading and the second reading and indicative of a level of blood perfusion improvement; and generating, with the machine learning model, an intervention recommendation indicative of whether additional intervention is recommended based on the first reading, the second reading, the intervention perfusion status, or combinations thereof.
10. The system of clause 9, wherein the first set of blood perfusion metrics and the second set of blood perfusion metrics include blood oxygenation, heart rate, bioimpedance, temperature, ankle-brachial pressure, or combinations thereof.
11. The system of any of clauses 9-10, wherein the plurality of sensors from the wearable device are positioned on the wearable device such that the plurality of sensors are positioned adjacent to the individual when the wearable device is worn by the individual.
12. The system of any of clauses 9-11, wherein: the first reading is a pre-intervention reading for establishing a baseline reading of blood perfusion; the second reading is an intervention reading for establishing a current reading of blood perfusion; and the intervention recommendation is a continuing intervention recommendation that includes an indication whether continued intervention should be provided, a predicted intervention perfusion status indicative of a predicted perfusion status result of the medical interventional at or after completion of the intervention, or combinations thereof.
13. The system of any of clauses 9-12, wherein: the first reading is a pre-intervention reading for establishing a baseline reading of blood perfusion; the second reading is a post-intervention reading for establishing a current reading of blood perfusion; and the intervention recommendation is a follow-up intervention recommendation that includes a recommended course of treatment, a predicted perfusion intervention status indicative of a predicted perfusion status result of the medical intervention within a time period following the medical intervention, or combinations thereof.
14. The system of any of clauses 9-13, wherein the machine-readable instructions cause the processor to perform operations further comprising: receiving a third set of blood perfusion metrics associated with the individual from the wearable device when the individual is wearing the wearable device; generating a third reading as a follow-up intervention reading after generation of the intervention recommendation and based on the third set of blood perfusion metrics; and training the machine learning model based on a comparison of the intervention recommendation and the third reading to improve subsequent follow-up intervention recommendations.
15. The system of clause 14, wherein the machine-readable instructions cause the processor to perform operations further comprising, before generating the intervention recommendation: receiving a historical data set including prior blood perfusion metrics, prior interventions, prior intervention statuses, or combinations thereof from a plurality of individuals; and training the machine learning model based on the historical data set to generate intervention status predictions based on blood perfusion metrics, interventions, intervention statuses, or combinations thereof.
16. A method for assessing blood perfusion, comprising: receiving, with a processor, a first set of blood perfusion metrics associated with an individual wearing a wearable device from the wearable device having a plurality of sensors for assessing blood perfusion; generating, with the processor, a first reading based on the first set of blood perfusion metrics; receiving, with the processor, a second set of blood perfusion metrics associated with the individual wearing the wearable device from the wearable device; generating, with the processor, a second reading based on the first set of blood perfusion metrics; determining an intervention perfusion status for improving blood perfusion for the individual based on the first reading and the second reading; and generating, with a machine learning model, an intervention recommendation indicative of whether additional intervention is recommended based on the first reading, the second reading, the intervention perfusion status, or combinations thereof.
17. The method of clause 16, wherein: the first reading is a pre-intervention reading for establishing a baseline reading of blood perfusion; the second reading is an intervention reading for establishing a current reading of blood perfusion; and the intervention recommendation is a continuing intervention recommendation that includes an indication whether continued intervention should be provided, a predicted intervention perfusion status indicative of a predicted perfusion status result of the medical intervention at or after completion of the intervention, or combinations thereof.
18. The method of any of clauses 16-17, wherein: the first reading is a pre-intervention reading for establishing a baseline reading of blood perfusion; the second reading is a post-intervention reading for establishing a current reading of blood perfusion; and the intervention recommendation is a follow-up intervention recommendation that includes a recommended course of treatment, a predicted intervention perfusion status indicative of a predicted perfusion status of the medical intervention within a time period following the medical intervention, or combinations thereof.
19. The method of any of clauses 16-18, further comprising: receiving a third set of blood perfusion metrics associated with the individual wearing the wearable device from the wearable device; generating a third reading as a follow-up intervention reading after generation of the intervention recommendation and based on the third set of blood perfusion metrics; and training the machine learning model based on a comparison of the intervention recommendation and the third reading to improve subsequent follow-up intervention recommendations.
20. The method of clause 19, further comprising, before generating the intervention recommendation: receiving a historical data set including prior blood perfusion metrics, prior interventions, prior intervention statuses, or combinations thereof from a plurality of individuals; and training the machine learning model based on the historical data set to generate intervention status predictions based on blood perfusion metrics, interventions, intervention statuses, or combinations thereof.

It should now be understood that embodiments disclosed herein are generally directed to wearable devices, systems, and methods for providing an assessment of blood perfusion. The wearable device, which may be a wearable garment, includes a plurality of sensors as well as a computing system for analyzing the data gathered by the plurality of sensors. The computing system may have machine-readable instructions executable by a processor for quantifying the perfusion of the body part wearing the wearable garment based on the gathered data. This, in turn, may make the determination of success of PAD interventions more objective. The machine-readable instructions may further include a machine learning model that is trained on sensor data for a variety of patients having PAD interventions of varying degrees of success. The machine learning model may further provide follow-up monitoring for determining perfusion success after intervention and enhancement of the machine learning model in future determinations and predictions.

It is noted that recitations herein of a component of the present disclosure being “configured” or “programmed” in a particular way, to embody a particular property, or to function in a particular manner, are structural recitations, as opposed to recitations of intended use. More specifically, the references herein to the manner in which a component is “configured” or “programmed” denotes an existing physical condition of the component and, as such, is to be taken as a definite recitation of the structural characteristics of the component.

The order of execution or performance of the operations in examples of the disclosure illustrated and described herein is not essential, unless otherwise specified. That is, the operations may be performed in any order, unless otherwise specified, and examples of the disclosure may include additional or fewer operations than those disclosed herein. For example, it is contemplated that executing or performing a particular operation before, contemporaneously with, or after another operation is within the scope of aspects of the disclosure.

Having described the subject matter of the present disclosure in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Further, it will be apparent that modifications and variations are possible without departing from the scope of the present disclosure, including, but not limited to, embodiments defined in the appended claims. More specifically, although some aspects of the present disclosure are identified herein as preferred or particularly advantageous, it is contemplated that the present disclosure is not necessarily limited to these aspects.

1. A system for assessing blood perfusion, comprising:
a wearable garment;
a plurality of sensors affixed to the wearable garment;
a processor communicatively coupled to the plurality of sensors;
a memory component communicatively coupled to the processor;
a machine learning model stored in the memory component; and
machine-readable instructions stored in the memory component that cause the processor to perform operations comprising:
receiving a first set of blood perfusion metrics associated with an individual wearing the wearable garment from the plurality of sensors;
generating a first reading based on the first set of blood perfusion metrics;
receiving a second set of blood perfusion metrics associated with the individual wearing the wearable garment from the plurality of sensors;
generating a second reading based on the second set of blood perfusion metrics;
determining an intervention perfusion status of a medical intervention to improve blood perfusion for the individual based on the first reading and the second reading and indicative of a level of blood perfusion improvement; and
generating, with the machine learning model, an intervention recommendation indicative of whether additional intervention is recommended based on the first reading, the second reading, the intervention perfusion status, or combinations thereof.

2. The system of claim 1, wherein the first set of blood perfusion metrics and the second set of blood perfusion metrics include blood oxygenation, heart rate, bioimpedance, temperature, ankle-brachial pressure, or combinations thereof.

3. The system of claim 1, wherein the plurality of sensors are positioned on the wearable garment such that the plurality of sensors are positioned adjacent to the individual when the wearable garment is worn by the individual.

4. The system of claim 1, wherein:
the first reading is a pre-intervention reading for establishing a baseline reading of blood perfusion;
the second reading is an intervention reading for establishing a current reading of blood perfusion during the medical intervention; and
the intervention recommendation is a continuing intervention recommendation that includes an indication whether continued intervention should be provided, a predicted intervention perfusion status indicative of a predicted perfusion status result of the medical intervention at or after completion of the intervention, or combinations thereof.

5. The system of claim 1, wherein:
the first reading is a pre-intervention reading for establishing a baseline reading of blood perfusion;
the second reading is a post-intervention reading for establishing a current reading of blood perfusion; and
the intervention recommendation is a follow-up intervention recommendation that includes a recommended course of treatment, a predicted intervention perfusion status indicative of a predicted perfusion status result of the medical intervention within a time period following the medical intervention, or combinations thereof.

6. The system of claim 1, wherein the machine-readable instructions cause the processor to perform operations further comprising:
receiving a third set of blood perfusion metrics associated with the individual wearing the wearable garment from the plurality of sensors;
generating a third reading as a follow-up intervention reading after generation of the intervention recommendation and based on the third set of blood perfusion metrics; and
training the machine learning model based on a comparison of the intervention recommendation and the third reading to improve subsequent follow-up intervention recommendations.

7. The system of claim 1, wherein the machine-readable instructions cause the processor to perform operations further comprising, before generating the intervention recommendation, receiving a historical data set including prior blood perfusion metrics, prior interventions, prior intervention statuses, or combinations thereof from a plurality of individuals.

8. The system of claim 7, wherein the machine-readable instructions cause the processor to perform operations further comprising, before generating the intervention recommendation, training the machine learning model based on the historical data set.

9. A system for assessing blood perfusion, comprising:
a processor;
a memory component communicatively coupled to the processor;
a machine learning model stored in the memory component; and
machine-readable instructions stored in the memory component that cause the processor to perform operations comprising:
receiving a first set of blood perfusion metrics associated with an individual from a wearable device having a plurality of sensors for assessing blood perfusion when the individual is wearing the wearable device;
generating a first reading based on the first set of blood perfusion metrics;
receiving a second set of blood perfusion metrics associated with the individual from the wearable device when the individual is wearing the wearable device;
generating a second reading based on the second set of blood perfusion metrics;
determining an intervention perfusion status for improving blood perfusion for the individual based on the first reading and the second reading and indicative of a level of blood perfusion improvement; and
generating, with the machine learning model, an intervention recommendation indicative of whether additional intervention is recommended based on the first reading, the second reading, the intervention perfusion status, or combinations thereof.

10. The system of claim 9, wherein the first set of blood perfusion metrics and the second set of blood perfusion metrics include blood oxygenation, heart rate, bioimpedance, temperature, ankle-brachial pressure, or combinations thereof.

11. The system of claim 9, wherein the plurality of sensors from the wearable device are positioned on the wearable device such that the plurality of sensors are positioned adjacent to the individual when the wearable device is worn by the individual.

12. The system of claim 9, wherein:
the first reading is a pre-intervention reading for establishing a baseline reading of blood perfusion;
the second reading is an intervention reading for establishing a current reading of blood perfusion; and
the intervention recommendation is a continuing intervention recommendation that includes an indication whether continued intervention should be provided, a predicted intervention perfusion status indicative of a predicted perfusion status result of the medical intervention at or after completion of the intervention, or combinations thereof.

13. The system of claim 9, wherein:
the first reading is a pre-intervention reading for establishing a baseline reading of blood perfusion;
the second reading is a post-intervention reading for establishing a current reading of blood perfusion; and
the intervention recommendation is a follow-up intervention recommendation that includes a recommended course of treatment, a predicted intervention perfusion status indicative of a predicted perfusion status result of the medical intervention within a time period following the medical intervention, or combinations thereof.

14. The system of claim 9, wherein the machine-readable instructions cause the processor to perform operations further comprising:
receiving a third set of blood perfusion metrics associated with the individual from the wearable device when the individual is wearing the wearable device;
generating a third reading as a follow-up intervention reading after generation of the intervention recommendation and based on the third set of blood perfusion metrics; and
training the machine learning model based on a comparison of the intervention recommendation and the third reading to improve subsequent follow-up intervention recommendations.

15. The system of claim 14, wherein the machine-readable instructions cause the processor to perform operations further comprising, before generating the intervention recommendation:
receiving a historical data set including prior blood perfusion metrics, prior interventions, prior intervention statuses, or combinations thereof from a plurality of individuals; and
training the machine learning model based on the historical data set to generate intervention status predictions based on blood perfusion metrics, interventions, intervention statuses, or combinations thereof.

16. A method for assessing blood perfusion, comprising:
receiving, with a processor, a first set of blood perfusion metrics associated with an individual wearing a wearable device from the wearable device having a plurality of sensors for assessing blood perfusion;
generating, with the processor, a first reading based on the first set of blood perfusion metrics;
receiving, with the processor, a second set of blood perfusion metrics associated with the individual wearing the wearable device from the wearable device;
generating, with the processor, a second reading based on the first set of blood perfusion metrics;
determining an intervention perfusion status for improving blood perfusion for the individual based on the first reading and the second reading and indicative of a level of blood perfusion improvement; and
generating, with a machine learning model, an intervention recommendation indicative of whether additional intervention is recommended based on the first reading, the second reading, the intervention perfusion status, or combinations thereof.

17. The method of claim 16, wherein:
the first reading is a pre-intervention reading for establishing a baseline reading of blood perfusion;
the second reading is an intervention reading for establishing a current reading of blood perfusion; and
the intervention recommendation is a continuing intervention recommendation that includes an indication whether continued intervention should be provided, a predicted intervention perfusion status indicative of a predicted perfusion status result of the medical intervention at or after completion of the intervention, or combinations thereof.

18. The method of claim 16, wherein:
the first reading is a pre-intervention reading for establishing a baseline reading of blood perfusion;
the second reading is a post-intervention reading for establishing a current reading of blood perfusion; and
the intervention recommendation is a follow-up intervention recommendation that includes a recommended course of treatment, a predicted intervention perfusion status indicative of a predicted perfusion status result of the medical intervention within a time period following the medical intervention, or combinations thereof.

19. The method of claim 16, further comprising:
receiving a third set of blood perfusion metrics associated with the individual wearing the wearable device from the wearable device;
generating a third reading as a follow-up intervention reading after generation of the intervention recommendation and based on the third set of blood perfusion metrics; and
training the machine learning model based on a comparison of the intervention recommendation and the third reading to improve subsequent follow-up intervention recommendations.

20. The method of claim 19, further comprising, before generating the intervention recommendation:
receiving a historical data set including prior blood perfusion metrics, prior interventions, prior intervention statuses, or combinations thereof from a plurality of individuals; and
training the machine learning model based on the historical data set to generate intervention status predictions based on blood perfusion metrics, interventions, intervention statuses, or combinations thereof.
